(19) United States
(12) Patent Application Publication
(10) Pub. No.: US 2026/0284143 A1
(43) Pub. Date: Sep. 24, 2026

(54) METHOD OF USING BIOACTIVE PEPTIDE FRACTION ISOLATED FROM CANARY SEED

(71) Applicant: Purdue Research Foundation, West Lafayette, IN (US)

(72) Inventors: Andrea M. Liceaga, West Lafayette, IN (US); Uriel Urbizo-Reyes, West Lafayette, IN (US)

(21) Appl. No.: 19/473,113

(22) PCT Filed: Apr. 3, 2024
(86) PCT No.: PCT/US2024/022830

§ 371 Date: Oct. 6, 2025

Related U.S. Application Data
(60) Provisional application No. 63/457,569, filed on Apr. 6, 2023.

(51) Int. Cl. A61K 38/16; A61K 36/899; A61P 3/04

(52) U.S. Cl. CPC A61K 38/168; A61K 36/899; A61P 3/04; A61K 2236/331; A61K 2236/51; A61K 2236/53

(57) ABSTRACT

A method of suppressing hunger in a human subject, who is obese or at risk for obesity (e.g., overweight), comprising administering at least about 20 mg/kg body weight/day of bioactive peptides comprising VPPR, LADR, LSPR, TVGPR, or any combination of two or more (e.g., two, three, or four) of the foregoing as a protein hydrolysate mixture, wherein the bioactive peptides can be obtained in a low molecule weight (<3 kDa) peptide fraction obtained from canary seed (Phalaris canariensis L.).

### CROSS REFERENCE TO RELATED APPLICATION

This application claims priority to U.S. provisional patent application No. 63/457,569, which was filed on Apr. 6, 2023.

### TECHNICAL FIELD

This disclosure relates to a bioactive peptide fraction isolated from Phalaris canariensis L. (“hairless canary seed”) and methods of use, e.g., to suppress hunger, inhibit weight gain, promote weight loss, improve glucose intolerance, and provide antioxidant activity.

### BACKGROUND

Obesity is a chronic metabolic disorder in which increased adiposity impairs physical and mental health. This condition has reached epidemic proportions globally and has become the major contributor to the global burden of non-communicable diseases (NCDs) [1]. During obesity, adipose tissue stores excess energy in adipocytes cells in the form of triglycerides. As adipose tissue expansion continues, it triggers the production of mediator molecules like adipocytokines or adipokines that give rise to chronic inflammation in various organs and cells [2]. As a risk factor, chronic inflammation is now recognized as an embedded mechanism in the deteriorating function of numerous physiological systems leading to unfavorable alteration in blood pressure, triglycerides, and insulin sensitivity [3]. Remarkably, evidence indicates that the regular consumption of plant-derived bioactive compounds (e.g., phenolic compounds, bioactive peptides (biopeptides), fatty acids, phytochemicals, and sterols) can lower the risk of developing obesity-related complications [4]. In this regard, biopeptides can offer anti-obesity properties via multiple mechanisms like interfering with macronutrient absorption, decreasing adipogenesis, modulating energy expenditure, suppressing appetite, reducing inflammation, and modifying intestinal micro-biota, among others [5, 6]. Moreover, it's been established that based on their composition, biopeptides can exert certain bioactivities due to their high specificity, affinity, and efficiency towards specific receptors and enzymes (e.g., α-glucosidase, angiotensin-converting en-zyme, α-amylase, and pancreatic lipase) [6]. For instance, soy peptides prevented obesity by activating a leptin-like signaling pathway [7], whereas cocoa peptides exhibit anti-obesity potential by inhibiting pancreatic lipase and preventing NCDs development [8]. Other studies on date seed (Phoenix dactylifera) protein hydrolysates showed the antidiabetic capacity of bromelain-generated digests due to their inhibitory activity towards dipeptidyl peptidase-IV (DPP-IV) and α-glucosidase [9].

Recently, hairless canary seed (Phalaris canariensis L.), an annual cereal grain, emerged as a novel and safe food ingredient with various nutritional and techno-functional attributes due to its small starch granules, phytochemical profile, and high protein content (19-24%) [10]. Interestingly, over decades, hairless canary seeds have been processed and employed as a traditional medicinal drink, known as “leche de alpiste” (alpiste milk) in Latin American countries to alleviate chronic conditions like cardiovascular diseases, diabetes, hypertension, oxidative damage, and obesity [11-13]. In vitro studies have associated the biological activities of canary seed with biopeptides produced during gastrointestinal digestion of the hydrophobic proteins present in the seed [13-15]. In this respect, research from our group has unveiled that canary seed peptides produced from commercial proteolysis with Alcalase® have high in vitro bioavailability and are remarkably stable as inhibitors of metabolism-regulating enzymes like pancreatic lipase, angiotensin-converting enzyme (ACE), and dipeptidyl peptidase-IV (DPP-IV) even after simulated gastrointestinal digestion [16]. Although some of the metabolic benefits (e.g., decrease in blood pressure, hypocholesterolemic properties, and hypoglycemic activity) of consuming whole canary seeds have been established in vivo [11, 12, 17], the studies remain limited, and there is no proposed mechanism of action nor identification of the biologically active compounds associated with the observed health benefits.

### SUMMARY

A method of suppressing hunger in a human subject, who is obese or at risk for obesity (e.g., overweight), is provided. The method comprises administering at least about 20 mg/kg body weight/day (e.g., 20 mg/kg body weight/day) of bioactive peptides comprising VPPR, LADR, LSPR, TVGPR, or any combination of two or more (e.g., two, three, or four) of the foregoing as a protein hydrolysate mixture, whereupon hunger in the subject is suppressed. The method can comprise administering at least about 20 mg/kg body weight/day (e.g., 20 mg/kg body weight/day) to about 40 mg/kg body weight/day (e.g., 40 mg/kg body weight/day) of bioactive peptides as a protein hydrolysate mixture. The bioactive peptides are present in a low molecular weight (<3 kDa) peptide fraction obtained from canary seed (Phalaris canariensis L.). The low molecular weight (<3 kDa) peptide fraction can be obtained from canary seed (Phalaris canariensis L.) by a method comprising:
(i) suspending defatted canary seed flour in distilled water at a pH of about 10 (e.g., pH=10) and stirring;
(ii) centrifuging to obtain a protein-rich supernatant;
(iii) adjusting the pH of the protein-rich supernatant to about 4 (e.g., pH=4);
(iv) centrifuging to precipitate protein;
(v) suspending the protein in distilled water at a pH of about 8 (e.g., pH=8);
(vi) subjecting the suspended protein to proteolysis with Alcalase® (protease of Bacillus licheniformis);
(vii) terminating proteolysis;
(viii) centrifuging to obtain a whole protein hydrolysate (WPH) as a supernatant; and
(ix) either (a) ultrafiltering the supernatant using a <3 kDa cutoff membrane to collect a peptide fraction comprising bioactive peptides, freezing, and freeze-drying or (b) subjecting the WPH to simulated gastrointestinal digestion (SGD), terminating SGD, centrifuging to obtain a supernatant, ultrafiltering the supernatant using a <3 kDa cutoff membrane to collect a peptide fraction comprising bioactive peptides, freezing, and freeze-drying. The bioactive peptides (as a protein hydrolysate mixture) can be administered before or with a meal comprising protein, wherein the combined amount of bioactive peptides (as a protein hydrolysate mixture) and protein is about 0.33 g/kg body weight/meal to about 0.40 g/kg body weight/meal (e.g., 0.33 g/kg body weight/meal to about 0.40 g/kg body weight/meal, about 0.33 g/kg body weight/meal to 0.40 g/kg body weight/meal, or 0.33 g/kg body weight/meal to 0.40 g/kg body weight/meal).

As a result of the administration, glucose tolerance in the subject can increase, weight gain can be inhibited, and the subject can lose weight. An added benefit of the method is antioxidant activity.

### FIGURES

FIG. 1. Effect of canary seed peptides (CSP) oral administration over body weight (FIGS. 1A-1B), weekly and daily feed intake (FIGS. 1C-1D), and intraperitoneal glucose tolerance in Western diet induced obese C57BL/6J mice at the seventh week of the study and prior to a six-hour fasting period (FIGS. 1E-1F). A weight per week (g) vs. time (weeks) (FIG. 1A); individual weight gain (g) vs. treatment (FIG. 1B); weekly food intake (g) vs. time (weeks) (FIG. 1C); daily feed intake (g/day) vs. treatment (FIG. 1D); blood glucose (mg/dL) vs. time (min) (FIG. 1E); and blood glucose (AUC) vs. treatment (FIG. 1F). ND, normal diet; WD, Western diet; CSP-250, mice supplemented with 250 mg/kg of body weight of CSP; CSP-500, mice supplemented with 500 mg/kg of body weight of CSP; Orlistat, mice supplemented with 40 mg/kg of body weight of orlistat. Bars and lines represent mean values=SEM (n=8 per treatment). Different letters (a-c) indicate statistical differences (p<0.05) between treatments.

FIG. 2. Effect of canary seed peptides (CSP) administration on serum biochemistry after eight weeks of diet-induced intervention in C57BL/6J mice. Fasting serum levels of triglycerides (A), total cholesterol (B), high density lipoprotein (C), and low/very-low density lipoprotein (D). ND, normal diet; WD, Western diet; CSP-250, mice supplemented with 250 mg/kg of body weight of CSP; CSP-500, mice supplemented with 500 mg/kg of body weight of CSP; Orlistat, mice supplemented with 40 mg/kg of body weight of orlistat. Bars and lines represent mean values±SEM (n=8 per treatment). Different letters (a-c) indicate statistical differences (p<0.05) between treatments.

FIG. 3. Effects of canary seed peptides (CSP) administration on regulation of metabolic hormones after eight weeks of diet-induced intervention in C57BL/6J mice and inhibition against pancreatic lipase (PL). Fasting serum levels of ghrelin (A), leptin (B), insulin (C), and adiponectin (D) hormones. Complete inhibition of PL by CSP and orlistat (E). 20-fold dilution of PL-inhibitor complex with oleate promotes recovery of PL activity from PL-CSP complexes (F). ND, normal diet; WD, Western diet; CSP-250, mice supplemented with 250 mg/kg of body weight of CSP; CSP-500, mice supplemented with 500 mg/kg of body weight of CSP; Orlistat, mice supplemented with 40 mg/kg of body weight of orlistat. Bars and lines represent mean values±SEM (n=8 per treatment). Different letters (α-d) indicate statistical differences (p<0.05) between treatments.

FIG. 4. (A) Triglycerides levels and (B) lipid metabolism related gene expression on hepatic tissue. ND, normal diet; WD, Western diet; CSP-250, mice supplemented with 250 mg/kg of body weight of CSP; CSP-500, mice supplemented with 500 mg/kg of body weight of CSP; Orlistat, mice supplemented with 40 mg/kg of body weight of orlistat. Bars and lines represent mean values±SEM (n=8 per treatment). Different letters (a-c) indicate statistical differences (p<0.05) between treatments. * p<0.05, ** p<0.05, #p<0.005, and ##p<0.0001 vs. WD group.

### DETAILED DESCRIPTION

The present disclosure is predicated, at least in part, on the results of the study of the effects of canary seed (Phalaris canariensis L.) peptides (CSP) on obesity in vivo. C57BL/6J mice were assigned into groups and fed a normal diet or a Western diet with an oral dose of CSP (250 (CSP-250) or 500 mg/kg/day (CSP-500)), orlistat (40 mg/kg/day), or distilled water. The results showed that consuming CSP can provide metabolic benefits, including preventing weight gain by up to 20%, increasing glucose tolerance, and reducing insulin, leptin, and LDL/VLDL levels in plasma. Conversely, total ghrelin was unaffected by CSP-500 and amplified by orlistat. Surprisingly, CSP-250 was more effective in preventing weight gain and promoting satiety than CSP-500. Parallel to this, protein absorption in CSP-500 was decreased, supported by a rise in fecal crude protein (3.5%). Similarly, fecal fat was increased by orlistat (38%) and was unaffected by CSP-250 (3.0%) and CSP-500 (3.0%), comparatively to a Western diet (2.5%). CSP-250 and CSP-500 were equally effective in decreasing hepatic steatosis and avoiding hyperlipidemia. Enzymatic analysis showed that CSP-pancreatic lipase (PL) complexes dissociated faster (15 minutes) than orlistat-PL complexes (41 minutes). Lastly, CSP did not affect expression of hepatic lipid oxidation genes ACO and PPAR-α but reduced the expression of the hydrolase gene LPL and lipogenesis-related genes FAS and ACC. These results indicate that a decrease in lipid metabolism increases fat transit time and, subsequently, suppresses hunger.

In view of the above, a method of suppressing hunger in a human subject, who is obese or at risk for obesity (e.g., overweight), is provided. The method comprises administering at least about 20 mg/kg body weight/day (e.g., 20 mg/kg body weight/day) of bioactive peptides comprising VPPR, LADR, LSPR, TVGPR, or any combination of two or more (e.g., two, three, or four) of the foregoing as a protein hydrolysate mixture, whereupon hunger in the subject is suppressed. The method can comprise administering at least about 20 mg/kg body weight/day (e.g., 20 mg/kg body weight/day) to about 40 mg/kg body weight/day (e.g., 40 mg/kg body weight/day) of bioactive peptides as a protein hydrolysate mixture. The bioactive peptides are present in a low molecular weight (<3 kDa) peptide fraction obtained from canary seed (Phalaris canariensis L.). The low molecular weight (<3 kDa) peptide fraction can be obtained from canary seed (Phalaris canariensis L.) by a method comprising:
(i) suspending defatted canary seed flour in distilled water at a pH of about 10 (e.g., pH=10) and stirring;
(ii) centrifuging to obtain a protein-rich supernatant;
(iii) adjusting the pH of the protein-rich supernatant to about 4 (e.g., pH=4);
(iv) centrifuging to precipitate protein;
(v) suspending the protein in distilled water at a pH of about 8 (e.g., pH=8);
(vi) subjecting the suspended protein to proteolysis with Alcalase® (protease of Bacillus licheniformis);
(vii) terminating proteolysis;
(viii) centrifuging to obtain a whole protein hydrolysate (WPH) as a supernatant, and
(ix) either (a) ultrafiltering the supernatant using a <3 kDa cutoff membrane to collect a peptide fraction comprising bioactive peptides, freezing, and freeze-drying or
   (b) subjecting the WPH to simulated gastrointestinal digestion (SGD), terminating SGD, centrifuging to obtain a supernatant, ultrafiltering the supernatant using a <3 kDa cutoff membrane to collect a peptide fraction comprising bioactive peptides, freezing, and freeze-drying. The bioactive peptides (as a protein hydrolysate mixture) can be administered before or with a meal comprising protein, wherein the combined amount of bioactive peptides (as a protein hydrolysate mixture) and protein is about 0.33 g/kg body weight/meal to about 0.40 g/kg body weight/meal (e.g., 0.33 g/kg body weight/meal to about 0.40 g/kg body weight/meal, about 0.33 g/kg body weight/meal to 0.40 g/kg body weight/meal, or 0.33 g/kg body weight/meal to 0.40 g/kg body weight/meal).

As a result of the administration, glucose tolerance in the subject can increase, weight gain can be inhibited, and the subject can lose weight. An added benefit of the method is antioxidant activity.

### EXAMPLES

The following examples serve to illustrate the present disclosure. The examples are not intended to limit the scope of the claimed invention in any way.

### Materials and Methods

### Preparation of Canary Seed Peptides (CSP)

USDA-GRAS hairless canary seeds (CDC Cibo) were purchased from a commercial vendor (Canpulse Foods LTD, Saskatoon, SK, Canada). As previously indicated, canary seeds were subject to mechanical oil removal [15]. Then, the defatted canary seed flour was subject to isoelectric protein extraction following the methodology proposed by Achouri et al. [45] with slight modifications. Briefly, defatted canary seed flour was suspended in distilled water (1:15 ratio, protein: liquid), and the pH was adjusted to 10 using 4 M NaOH, followed by constantly stirring for one hour at 22±3° C. The protein-rich fraction (supernatant) was recovered by centrifugation at 1000×g for 20 min, and then the pH was adjusted to 4 using 1 N HCl. A second centrifugation step was used to precipitate the canary seed protein. The isolated protein was then suspended in distillated water (22.5 mg of protein/mL at pH 8) and subject to proteolysis [16]. The reaction was carried out at 50±3° C. for four hours with 3% (w/w) food-grade Alcalase® (Protease of Bacillus licheniformis, P2.4 U/g; Novozymes, Bagsvaerd, Denmark). Proteolysis was terminated by pasteurization (95±3° C.) for 15 min. Canary seed peptides were recovered from the supernatant by centrifugation (17,636 g for 15 min) (Avanti J-26S Centrifuge, Beckman-Coulter Inc., Brea, CA, USA), frozen (−80° C.), and lyophilized using a Labconco FreeZone Plus 2.5 μL cascade benchtop freeze dry system (Labconco Corp., Kansas City, MO, USA). Canary seed peptide powder was composed of 70% crude protein, 1.3% crude fat, 17% carbohydrate, and 11.4% ash dry basis. Finally, CSP's proximate composition was determined, as we previously described [46], through a commercial analytical laboratory (A&L Great Lakes, Fort Wayne, IN, USA).

### Animals and Housing

Four-week-old male C57BL/6J mice (n=40) were purchased from the Jackson Labora-tory (Bar Harbor, ME, USA). All the experimental animal procedures were approved by Purdue University Animal Care and Use Committee [PACAUC approval No. 1810001817A003] according to the recommendations of the Guide for the Care and Use of Laboratory Animals published by the National Institutes of health. The mice had ad libitum access to water and food and were housed under 12 h light/dark cycles at 23±3° C. and controlled humidity 50-60% throughout the experimental period.

### Experimental Design, Diets, and Sample Collection

Following arrival, mice were acclimatized for ten days and then randomly allocated into the following five groups (n=8 per group): normal diet (ND) group, mice were fed a standard diet+saline solution; Western diet (WD) group, mice were fed a western-style diet+saline solution; canary seed peptide-250 (CSP-250) and 500 (CSP-500) groups, mice were fed a western-style diet+250 or 500 mg CSP/kg of body mass solution; and orlistat group, mice were fed a western-style diet+40 mg orlistat/kg of body mass solution. Distilled water, CSP, and orlistat solutions were supplemented daily in equivalent volumes by oral gavage for the eight weeks of the study. The standard diet (TD.05230, with 4% of fat calories) and Western-style diet (TD.88137, with 42% of fat calories) were purchased from Envigo (Madison, WI, USA), and their formulation and caloric composition are given in Table 3.

TABLE 3
Proximal composition (wet basis) and
formulation of experimental diets

| | Normal Diet | Western Diet |
|---|---|---|
| Macro-nutrient | TD.05230 | TD.88137 |
| Protein (%) | 17.3 | 17.3 |
| Carbohydrate (%) | 63.5 | 48.5 |
| Fat (%) | 5.2 | 21.2 |
| Energy (kcal/g) | 3.7 | 4.5 |

| | |
|---|---|
| Ingredient | Formula (g/kg) |

| | | |
|---|---|---|
| Casein | 195 | 195 |
| DL-methionine | 3 | 3 |
| Sucrose | 341 | 341 |
| Corn Starch | 212 | 150 |
| Maltodextrin | 100 | n/a |
| Anhydrous milkfat | 37 | 210 |
| Soybean oil | 13 | n/a |
| Cholesterol | n/a | 1.5 |
| Cellulose | 50 | 50 |
| Mineral mix (AIN-76) | 35 | 35 |
| Calcium carbonate | 4 | 4 |
| Vitamin mix (Teklab-40060) | 10 | 10 |
| Ethoxyquien-antioxidant | 0.01 | 0.04 |

The total body weight gain (g/animal), daily feed intake (g/animal/day), and water consumption was monitored weekly throughout the study. The fecal collection was done per cage, separating fecal matter from bedding on a weekly basis and then stored at −80±3° C. until analysis. After the eight weeks of the study, animals were sacrificed by CO₂ asphyxiation. Immediately after, blood was collected by cardiac puncture. Finally, kidney, spleen, liver, and epididymal fat were weighted and stored at 80±3° C.

### Intraperitoneal Glucose Tolerance Test (IPGTT)

The glucose tolerance test was conducted at the seventh week of the study, following the previously described methodology by Small et al. [47]. Briefly, each mouse underwent a fasting period of six hours. Then, blood samples were collected to quantify circulating glucose by making a small incision at the tip of the tail and analyzed using a NovaMax glucometer (Nova Diabetes Care, Billerica, MA, USA). Blood glucose was quantified at baseline and after (30, 60, and 120 min) an intraperitoneal injection of a 45% D-(+)-Glucose solution (2 g of glucose/kg body mass). Finally, the results were analyzed and reported as an area under the curve (AUC).

### Fecal Proximal Composition

Moisture, crude protein, crude fat, crude fiber, of the fecal matter were determine by the official methods of analysis AOAC 934.01, AOAC 990.03, AOCS Am5-04, AOCS Ba6a-05 by a commercial laboratory, Waters Agricultural Laboratories (Camilla, GA, USA).

### Serum Biochemical Analysis

Total serum high-density lipoprotein (HDL), low-density lipoprotein (LDL)/very low-density lipoprotein (VLDL), and total cholesterol levels were quantified with HDL, and LDL/VLDL Colorimetric/Fluorometric assay Kit (LSK314100) obtained from Lifespan Bioscience (Seattle, DC, USA). The assays were carried out following the manufacturer's instructions, and the absorbance was measured using Multiskan™ FC Microplate Photometer (Waltham, MA, USA). In addition, serum triglyceride (TG) levels were measured using a triglyceride colorimetric kit (LSK302200) purchased from Lifespan Bioscience (Seattle, DC, USA).

### Measurement of Fasting Serum Levels of Metabolic Hormones

Mouse serum was analyzed to quantify total serum ghrelin using a mouse enzyme-linked immunosorbent assay (ELISA) ghrelin kit (EIAM-GHR-1) obtained from Raybiotech (Norcross, GA, USA), serum leptin by mouse leptin Duoset ELISA kit (DY49805) obtained from R&D Systems (Minneapolis, MN, USA), serum adiponectin and insulin by commercial mouse ELISA kits (KMP0041 and EMINS) obtained from Thermo Fisher Scientific (Waltham, MA, USA). All assays were carried out following the manufacturer's instructions. Finally, the absorbances were quantified at 450 nm and turbidity corrections at 590 nm using Multiskan™ FC Microplate Photometer (Waltham, MA, USA).

### Pancreatic Lipase Inhibition

Pancreatic lipase inhibition was measured according to the procedures described in our previous study [16]. Briefly, samples, substrate, and pancreatic lipase were dis-solved in McIlvaine buffer (0.1 M Citrate-Na2HPO4, pH 7.4). Then, 50 μL of substrate 4-methylumbelliferyl oleate (1 mM) was pre-incubated with 25 μL of peptide sample for 10 min in a black 96-well microplate. Then, 25 μL of pancreatic lipase type-VI (200 U/mL) were pipetted, and the reaction was carried out for one hour. The production of 4-methylumbelliferone was measured fluorometrically using a Fluoroskan Ascent FL Microplate Fluorometer and Luminometer, ThermoFisher Scientific (Waltham, MA, USA) using an excitation wavelength of 355 nm and emission wavelength of 460 nm. The positive control (no inhibitor) used the buffer instead of the sample, and buffer was used instead of pancreatic lipase solution for negative control (no pancreatic lipase activity). Finally, a jump dilution experiment was carried out, as previously reported by Kumar and Lowery [48]. Briefly, enzyme-inhibitor complexes were formed by pre-incubating the substrate, pancreatic lipase (6000 U/mL), and inhibitors (10×IC50) for three hours. Then, the activity recovery was measured after performing a 30-fold jump dilution, and the reaction was monitored for 25 min. Finally, the residence time was calculated by measuring tau determined as the reciprocal of k_{off} after fitting the enzyme progress curves to Equation (1).

$$Abs = {V}_{s} t + ( {V}_{0} - {V}_{s} ) ( 1 - e - {k}_{offt} ) {k}_{off} \qquad ( Equation 1 )$$

### Histopathology

Liver sections were embedded in paraffin blocks, sliced [4 μm], and stained using standard hematoxylin and eosin staining, followed by microscopically evaluation. Similarly, oil red 0 staining was carried out by embedding in optimal cutting temperature gel, and the samples were cut to a thickness of 6 μm sections. The sections were quickly submerged in formalin and stained with 0.5% oil red 0 solution. All sides were prepared and evaluated by a board-certified veterinary pathologist from the Histology Research Lab-oratory at Purdue University. The hepatic histological scores were achieved by following the micromorphological scale.

### Hepatic Triglyceride Analysis

Triglycerides from mouse liver were extracted and quantified as proposed by Adhyatmika et al. [49], and Smith et al. [50]. Briefly, hepatic tissue was homogenized for 40 s in 1 mL of extraction buffer that contained 25 mM Tris, 10 mM sodium phosphate, 150 mM NaCl, 0.1% SDS, 1% Triton-X100, and protease inhibitor. The tissue sample was then diluted with 5 mL of a chloroform/methanol (2:1, v/v) solution vortex briefly and incubated under an ice bath for two hours. The solution was centrifuged at 1650×g for 10 min at 4° C., and three phases were identified. The upper phase was collected and subject to a second extraction step by adding 600 μL of 4 mM MgCl₂ and vortexed vigorously and incubated under an ice bath for 30 min, followed by centrifugation 1650×g for 10 min at 4° C. The bottom phase from the first extraction was combined with the bottom phase of the second extraction. The chloroform was removed by evaporation under nitrogen gas in a 37° C. water bath. The triglycerides were suspended with 200 μL of distillate water and vortexed vigorously to create the final emulsion. Finally, the triglyceride content was quantified using the Infinity Triglyceride Reagent (Thermo Scientific, Waltham, MA, USA) following manufacturer's instructions and normalized to protein content.

Gene Expression by Quantitative Real-Time Polymerase Chain Reaction (qPCR)

RNA was extracted following the methodology proposed by Siersbok et al. [51], and qRT-PCR analysis following the proposed method by Pfohl et al. [52]. Briefly, 15 mg of liver tissue were homogenized using TRIzol-RNA lysis reagent (Invitrogen) for 5 min. The RNA was then purified by adding 150 μL of chloroform followed by centrifugation at 12,000×g for 15 min at 4° C. The supernatant was recovered as a source of RNA and was further washed and centrifuged three times with 0.5 mL of 75% ethanol at 12,000×g for 3 min. Lastly, the pellet was resuspended with 0.5 mL of DPEC water, 0.5 mL of isopropanol, and 50 μL of a 3 M sodium acetate solution at pH 5.5 followed by centrifugation at 12,000×g for 15 min at 4° C. The precipitated RNA was then suspended in Diethyl pyrocarbonate (DEPC) water, and complementary DNA (cDNA) synthesis using the superscript II kit according to the manufacturer's protocol. The cDNA was amplificated using SYBR premix Plus (SYBR Green) (Bio-Rad Laboratories, Hercules, CA, USA). The reaction was carried out using StepOne Real-Time PCR System (Applied Biosystems, Foster City, CA, USA), and the expression was normalized to the housekeeping gene GAPDH. Lastly, the data were analyzed using the Delta Delta CT (2^{-ΔΔCt}) method to calculate the fold gene expression change relative to the control (ND).

### Statistical Analysis

The results in this study were analyzed using a randomized complete block design by a one-way-ANOVA followed by a Tukey's and Fisher's LSD multiple comparison test (p<0.05), where blocks were animal cages. Data analysis was done using the statistical software statistical software SAS® version 9.4 (SAS Institute, Cary, NC, USA) and GraphPad Prism® version 9.4.0 (GraphPad, San Diego, CA, USA). Finally, the results were reported as mean±standard error of the mean (SEM).

### Example 1. Effect of Canary Seed Protein (CSP) on Body Weight, Feed Consumption, and Glucose Tolerance in Mice

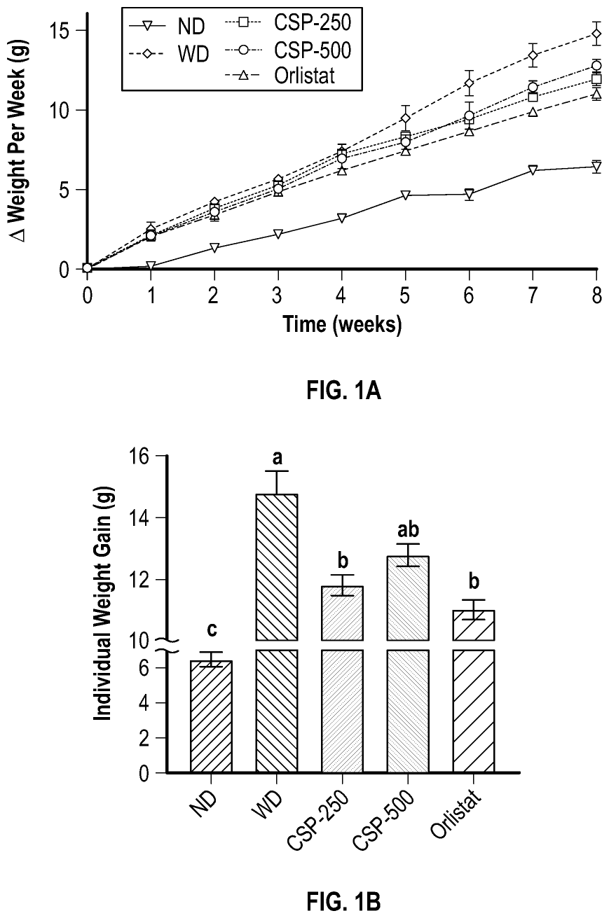

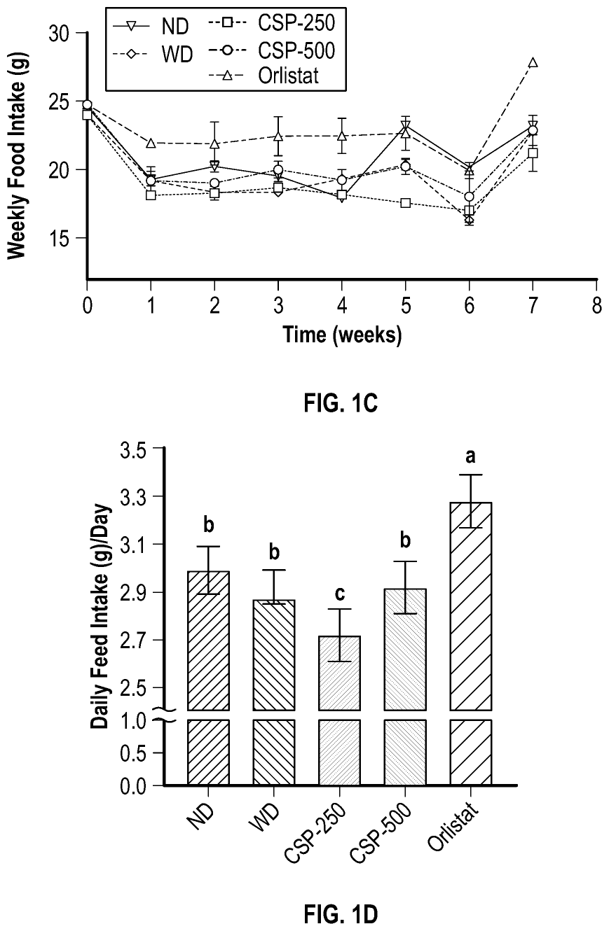

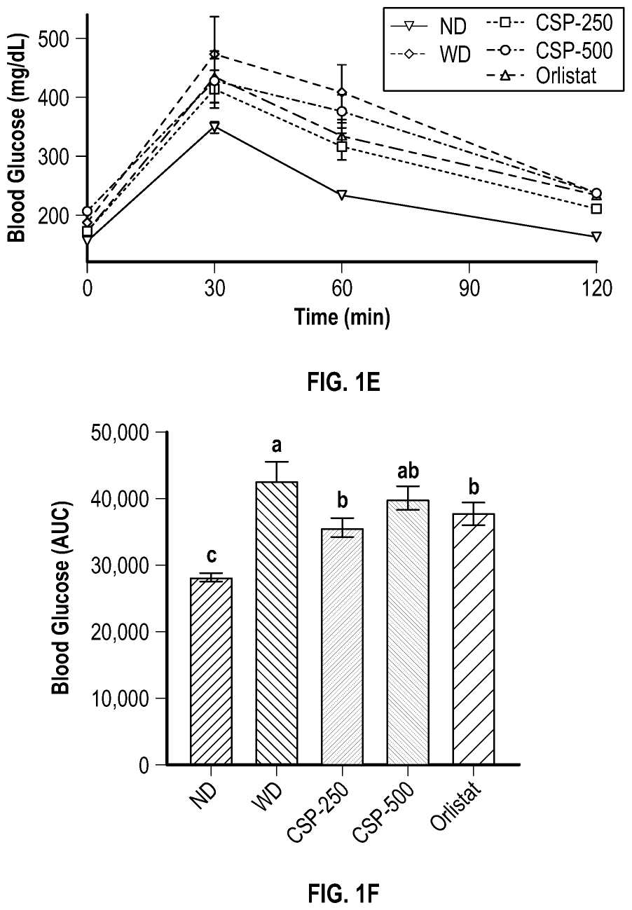

The proximate composition of the CSP powder used in this study comprised of 69.0% crude protein, 18% carbohydrate, 11.4% ash, and 1.3% fat (w/w, dry basis). In previous studies, we determined that this CSP powder is predominantly rich in low molecular weight peptides with a high degree of hydrolysis (>30%) [15,16]. After eight weeks of study, the daily supplementation of CSP-250 and orlistat, decreased (p<0.05) the body weight gain of mice by 20 and 26%, respectively (FIG. 1A). There was no significant (p<0.05) difference in weight gain prevention between CSP-250, CSP-500, and the synthetic drug orlistat. However, at the higher CSP doses (i.e., CSP-500), no difference (p>0.05) in weight gain was found compared to animals fed the Western diet (WD), indicating a drop in bioactivity at increasing peptide concentrations. As expected, mice fed the normal diet (ND) gained the least body weight after the culmination of the study. Likewise, the feed consumption was also affected, where the highest (p<0.05) daily feed intake (FIG. 1D) was observed in the mice supplemented with orlistat (3.3 g/day), and no difference was found between ND (3.0 g/day), CSP-500 (2.9 g/day), and WD (2.9 g/day). Interestingly, the mice supplemented with CSP-250 had the lowest (p<0.05) daily feed intake (2.7 g/day) throughout the study. These results indicate that at low CSP supplementation levels, CSP might also modulate feed intake.

The impact of CSP supplementation on glucose metabolism was assessed by monitoring blood glucose after an intraperitoneal glucose tolerance test (FIG. 1E) and determining the mean of the integrated area under the curves (AUC) (FIG. 1F). Mice on the WD suffered glucose intolerance, whereas mice under ND displayed the lowest post-glucose challenge AUC values. The AUC for mice treated with CSP-250 or orlistat was lower (p<0.05) than that observed for mice on the WD, but higher than mice fed the ND. The glucose tolerance of animals supplemented with higher peptide concentrations (CSP-500) was similar to CSP-250 and orlistat; however, no difference (p>0.05) was found when compared to mice in the WD group.

### Example 2. Effect of CSP on Lipid Profiles, Organ Weight, and Fecal Proximal Composition

To examine whether the weight gain inhibition of CSP supplementation was associated with adiposity and lipid metabolism, the organ weight and fecal composition were determined at the end of the study (Tables 1 and 2). The relative liver and epididymal adipose tissue weights were higher (p<0.05) for mice fed the WD and the lowest (p<0.05) for mice fed the ND. CSP dosages and orlistat successfully lowered relative hepatic weight and epididymal fat deposition to levels similar (p>0.05) to those mice in the ND group. Additionally, the kidney and spleen weights were also evaluated to establish any possible toxic response, and no difference (p>0.05) was found between the CSP, orlistat treatment, and the controls. The fecal composition analysis showed that orlistat supplementation resulted in the highest (p<0.05) fecal fat concentration. In contrast, excreted fecal fat from CSP-supplemented animals was not significantly (p>0.05) different from those of ND and WD groups. Furthermore, crude protein concentration in the fecal matter was significantly (p<0.05) increased by CSP-500 supplementation but not by CSP-250 compared to that of ND and WD animals. Additionally, the fecal concentrations of crude protein, crude fiber, and ash were the lowest for the mice supplemented with orlistat (Table 1).

TABLE 1
Proximal composition of fecal matter from C57BL/6J mice after 8 weeks of treatment

| | Fecal proximal composition (% in dry weight) | | | | |
|---|---|---|---|---|---|
| Treatment | Crude | | | Nitrogen-Free | |
| Group | Protein | Crude Fat | Crude Fiber | Extract | Ash |
| Normal diet | 17.64 ± 0.04 ab | 2.23 ± 0.30 b | 33.08 ± 0.93 a | 47.48 ± 0.93 b | 20.27 ± 0.63 a |
| Western diet | 15.29 ± 0.05 c | 2.58 ± 0.01 b | 28.9 ± 1.21 a | 47.56 ± 1.21 b | 21.85 ± 0.16 a |
| CSP250 | 16.14 ± 0.01 bc | 3.03 ± 0.01 b | 30.41 ± 1.06 a | 48.02 ± 1.06 b | 21.04 ± 0.41 a |
| CSP500 | 18.83 ± 0.91 a | 3.02 ± 0.39 b | 31.58 ± 0.80 a | 48.74 ± 0.80 b | 20.34 ± 0.63 a |
| Orlistat | 12.62 ± 0.15 d | 38.67 ± 0.16 a | 19.9 ± 0.32 a | 93.42 ± 0.32 a | 13.1 ± 0.19 b |

Values represent mean values ± SEM (n = 8 per treatment).
Different letters (a-d) within a column indicate statistical differences (p < 0.05) between treatments.
Bwt: body weight.

TABLE 2
Organ and epididymal fat weight for C57BL/6J mice after 8 weeks of treatment

| | Organ and Tissue Weights | | | |
|---|---|---|---|---|
| Treatment | Kidney | Spleen | Liver | Epididymal Fat |
| Group | (mg) | (mg) | (%, w/bwt) | (%, w/bwt) |
| Normal diet | 371.64 ± 4.36 a | 86.27 ± 3.50 a | 4.03 ± 0.06 b | 3.07 ± 0.27 d |
| Western diet | 390.76 ± 0.76 a | 85.25 ± 0.50 a | 5.42 ± 0.48 a | 6.68 ± 0.20 a |
| CSP250 | 386.50 ± 10.25 a | 80.4 ± 0.93 a | 4.70 ± 0.01 ab | 5.97 ± 0.12 bc |
| CSP500 | 369.08 ± 12.83 a | 84.60 ± 3.70 a | 4.61 ± 0.25 ab | 6.28 ± 0.45 ab |
| Orlistat | 402.08 ± 7.43 a | 81.50 ± 0.50 a | 4.46 ± 0.29 ab | 5.41 ± 0.32 c |

Values represent mean values ± SEM (n = 8 per treatment).
Different letters (a-d) within a column indicate statistical differences (p < 0.05) between treatments.
Bwt: body weight.

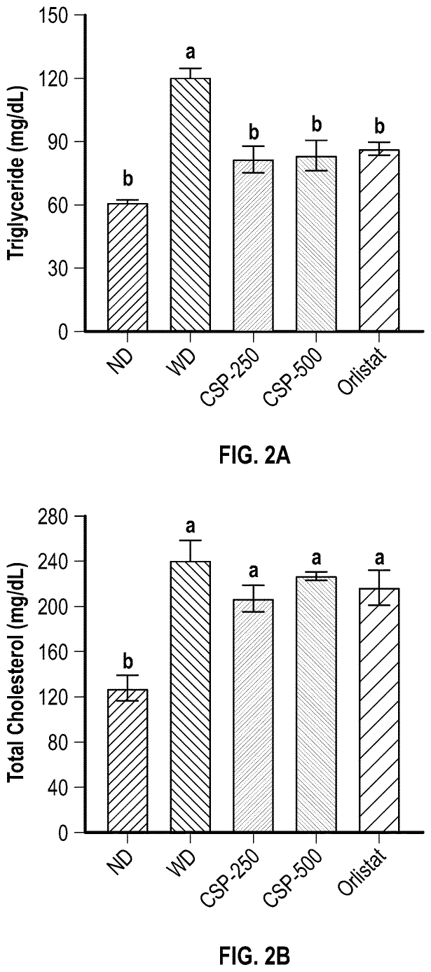

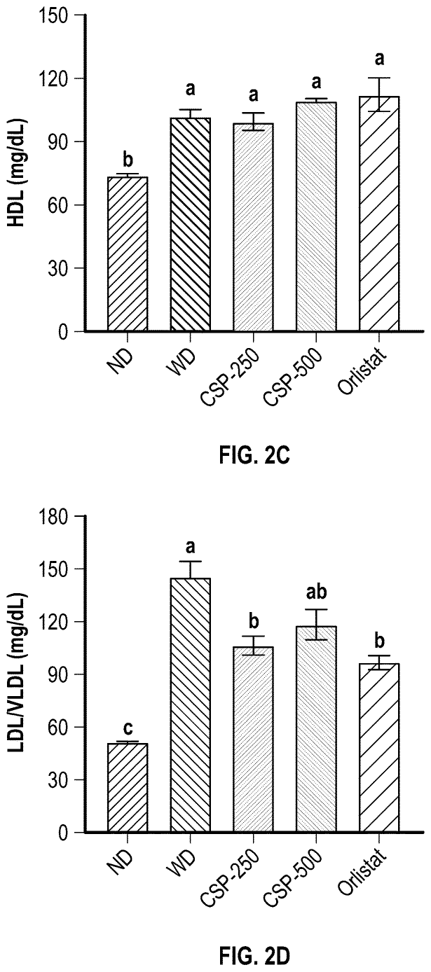

The serum TG, HDL, LDL/VLDL, and total cholesterol levels were the highest (p<0.05) in the WD group and lowest for the ND group (FIG. 2). In addition, CSP-250, CSP-500, and orlistat supplementation were effective in lowering (p<0.05) TG concentration in serum to similar levels of mice in the ND group (FIG. 2A). Similarly, CSP-250 and orlistat groups displayed a reduction (p<0.05) in the serum LDL/VLDL concentration compared to WD but were still higher than the ND group. Conversely, no significant difference (p>0.05) was found in LDL/VLDL serum concentrations between CSP-500 and WD groups. Likewise, no difference (p>0.05) in HDL and total cholesterol was found between CSP-250, CSP-500, orlistat, and the WD group. These results indicate that daily CSP supplementation can be an effective alternative for preventing hyperlipidemia and improving blood cholesterol profiles.

### Example 3. Modulatory Effect of CSP on Metabolic Hormone Levels and Pancreatic Lipase Activity

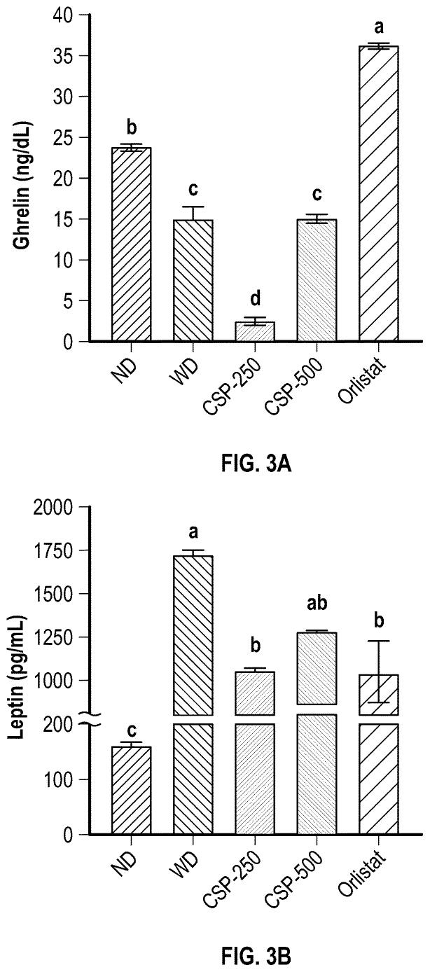

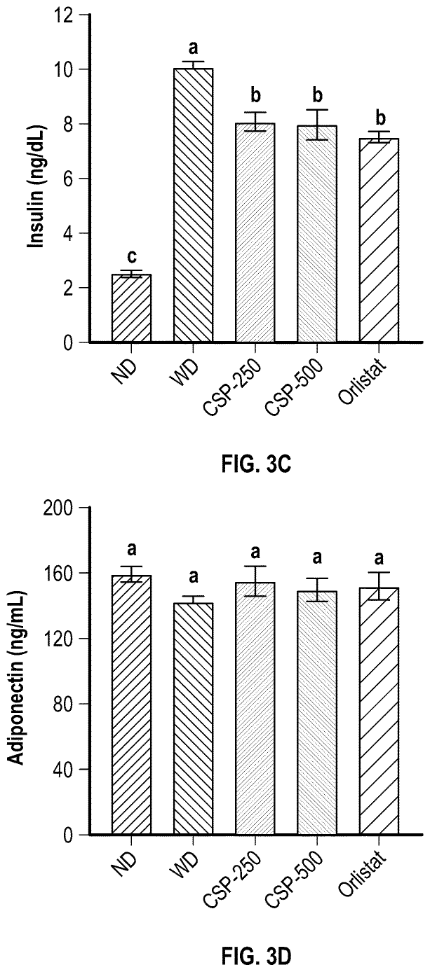

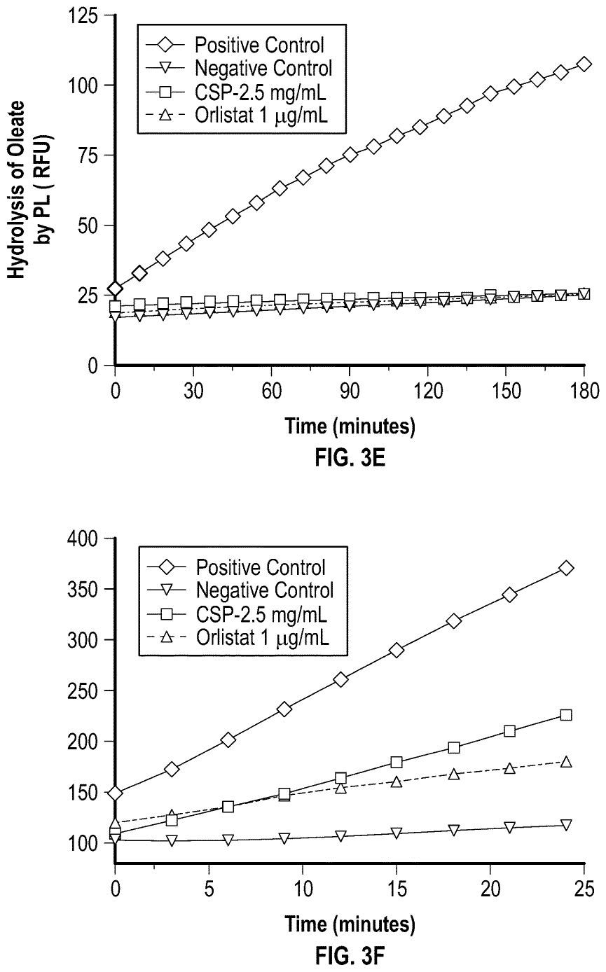

The effect of CSP on metabolic hormones and pancreatic lipase activity is shown in FIG. 3. At the end of the study, serum leptin and insulin levels were the highest (p<0.05) for the WD group and the lowest for mice under ND. Meanwhile, animal groups supplemented with CSP-250 and orlistat exhibit a reduction (p<0.05) in leptin and insulin levels. Additionally, when compared to the WD group, CSP-500 also decreased insulin levels significantly (p<0.05) but not leptin concentrations (p>0.05). Interestingly, CSP-250 supplementation, but not CSP-500, induced a significant (p<0.05) reduction of serum ghrelin concentrations compared to the controls. Conversely, orlistat supplementation increased (p<0.05) the concentration of serum ghrelin. These results aligned with the feed intake observations reported earlier, were animals in the CSP-250 diet group displayed the lowest daily feed intake and animals from the orlistat group had the highest daily feed intake (FIG. 1B).

The regulation of pancreatic lipase by CSP and orlistat is shown in FIGS. 3E and 3F, respectively. The pancreatic lipase-inhibitor complexes were pre-formed to inhibit the enzyme activity by completely saturating the enzyme with 10×IC₅₀ based on our previous studies [15, 16]. The complexes were stable and inactive (FIG. 3E), presenting no product formation, and were monitored for three hours. The residence time of the inhibitors was determined after performing a 30-fold jump dilution experiment with 4-methylumbelliferyl oleate. The results showed rapid recovery of pancreatic lipase activity by CSP inhibitor dissociation and a slow recovery in pancreatic lipase activity from orlistat complexes (FIG. 3F). The residence times for CSP and orlistat were 15 and 41 min, respectively. These results demonstrate the reversible inhibitory nature of CSP towards pancreatic lipase and the slow reversibility of orlistat once bound to pancreatic lipase.

### Example 4. Effect of CSP on Liver Histopathology, Lipid Metabolism, and Gene Expression

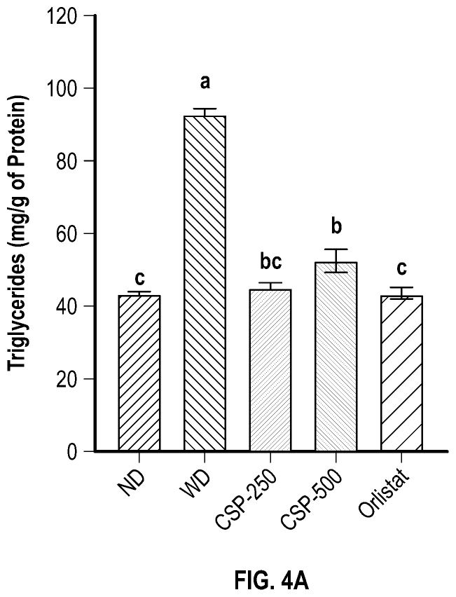

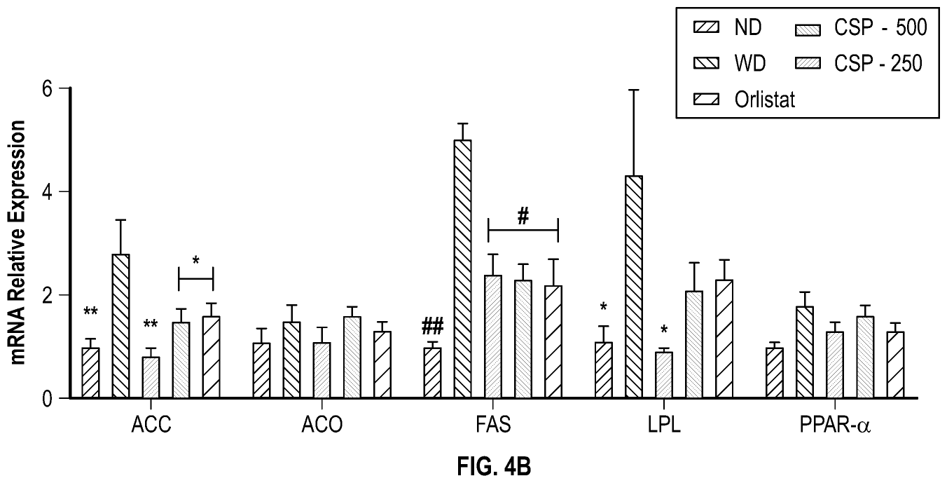

To further explain the changes in liver weight between treatments (CSP and orlistat) and WD reported in Table 2, and to further explore the protective effect of CSP in liver pathophysiology, histology, TG, and gene expression analysis was carried out (FIG. 4). TG analysis revealed that both CSP dosages and orlistat were successful in reducing (p<0.05) the hepatic TG deposition (steatosis) and had similar levels to those of mice in the ND group. These observations were further confirmed by microscopic examination of the liver tissue under Oil red O staining for neutral lipids. Two conditions became apparent: macrovesicular steatosis (large TG droplet vacuole filling the hepatocyte) in the WD group and microvesicular steatosis (small intracytoplasmic TG vacuole filling the hepatocyte) in CSP and orlistat treatments. No difference (p>0.01) was observed in the inflammatory parameters such as ballooning and lobular inflammation when evaluated microscopically under H&E staining.

In addition, hepatic gene expression analysis (FIG. 4B) showed a parallel trend in lipid metabolism genes between mice in the CSP and orlistat diet groups. Particularly, CSP and orlistat supplementation resulted in significant downregulation of fatty acid synthesis genes such as FAS and ACC. Meanwhile, genes involved in lipid oxidation (ACO and PPAR-α) remained unchanged compared to the WD group. Moreover, only the ND and CSP-250 diet groups exhibited a significant suppression in the expression of the LPL encoding gene.

### Example 5. Preparation of Low Molecular Weight (<3 kDa) Peptide Fraction from Canary Seeds

Canary seeds were prepared as previously reported [54]. Briefly, a canary seed protein solution (22.5 mg of protein/mL at pH 8) was hydrolyzed at 50±3° C. for four hours with 3% (w w) Alcalase®. Proteolysis was stopped by pasteurization (95±3° C.) for 15 min. Then, the solution was cooled and centrifuged (17,636×g for 15 min) (Avanti J-26S Centrifuge, Beckman-Coulter Inc., Brea, CA, USA). The supernatant was collected as a whole protein hydrolysate (WPH) and stored at −80° C. until use. The WPH was subject to simulated gastrointestinal digestion (SGD) using the method described by You, Zhao [55]. A WPH solution (10 mg of protein/mL, at pH 2) was prepared using 1 M HCl and then incubated with pepsin (4% weight/weight of protein) at 37° C. for two hours. The pH was increased to 5.3 using a 0.9 M NaHCO₃ solution and increased further to 7.5 using a 1.0 M NaOH solution. Pancreatin was then added (4% w/w of protein), and the mixture was incubated again at 37° C. for two hours. SGD was terminated by pasteurization (95±3° C.) for 15 min. Subsequently, the solution was centrifuged at 11,000×g for 15 min. The supernatant was ultrafiltered using a <3 kDa cutoff membrane to collect the peptide fraction identified in our previous study as being the most bioactive [54], which was referred to as canary seed peptide fraction (CSPF). CSPF was frozen at −80° C. for 12 h and freeze-dried using a Labconco FreeZone Plus 2.5 μL cascade benchtop freeze dry system (Labconco Corp., Kansas City, MO, USA).

In previous in vitro studies, it was established that CSP had a high potency in inhibiting pancreatic lipase and that, after gastrointestinal digestion, its potency increased 10-fold [15,16]. In addition, it was previously shown that CSP inhibited pancreatic lipase uncompetitively by destabilizing the open-lid conformation via arginine and non-polar residues interactions [16]. It is worth mentioning that CSP is less potent and stable than synthetic pancreatic lipase inhibitors like orlistat. Therefore, higher concentrations of CSP were tested to elucidate any possible biological effects in this study. Henceforth, the present study explored the potential therapeutical benefits of CSP consumption in preventing obesity and its metabolic consequences. The results demonstrated that C57BL/6J mice fed a WD and supplemented daily with CSP exhibited lower weight gain, decreased feed intake, and increased glucose tolerance compared to mice fed the WD alone. Furthermore, the CSP metabolic benefits observed in this study were similar to those of the commercial anti-obesity drug orlistat. In this respect, it is recognized that orlistat effectively inhibits gastric and pancreatic lipases, preventing the breakdown of dietary fats into absorbable triglycerides, free fatty acids, and monoglycerides, hence stopping fat absorption and increasing the fecal fat concentration [18-20]. Yet, it's been reported that undesired side effects might accompany treatment with synthetic drugs like orlistat (e.g., oily spotting, abdominal cramping, fecal incontinence, and fat-soluble vitamin deficiencies) [21]. In this study, CSP's potency was inferior to that of the commercial drug orlistat; however, the side effects like steatorrhea were not triggered by the peptides. Hence, studying natural inhibitors (e.g., polyphenols, peptides, flavonoids, saponins, terpenoids, alkaloids) from plants with fewer side effects is of high interest in nutraceutical research [22].

Surprisingly, this study showed that CSP-250 was more effective at preventing weight gain than CSP-500, with the latter being not different from the WD group. Likewise, CSP-250 but not CSP-500 decreased feed intake and ghrelin expression, yet both CSP treatments exhibited high hypolipidemic and anti-steatosis effects. Moreover, the drop in weight gain prevention capacity by CSP-500 could be attributed to the saturation of tolerable dietary protein intake per meal in the small intestine, leading to indigestion of the peptides. While no studies have yet suggested a maximum absorbable protein load (protein content/kg of BWT/meal, wherein BWT=body weight) in mice, recent research in humans suggests that optimal protein consumption should range between 0.33-0.40 g of protein/kg of BWT/meal and that higher protein levels may result in a rise in amino acid oxidation, and indigestion of proteins causing an increase in fecal protein concentration [23-25]. Other studies in pigs have reported similar observations, where a significant reduction of protein digestibility and an increase of fecal crude protein resulted from increased dietary protein levels' supplementation from 12 to 18% in ad libitum diets [26]. In agreement with this, the protein content in fecal matter from CSP-500 treated animals was higher than that of the controls and CSP-250 animals. However, additional studies are needed to fully understand the maximum tolerable protein load per meal/dosage in mice and the effect of CSP-500 when distributed into several doses throughout the day.

Considering that CSP-250 displayed hypolipidemic activity, satiety properties, and an absence of steatorrhea, this suggests that CSP-250 functioned through a dual mechanism to prevent obesity. It is hypothesized that a delay in lipid metabolism might account for the satiety and hypolipidemic observations in the CSP-250 animals. The enzymatic study revealed that the residence time of CSP over pancreatic lipase enzyme was 2.7-fold shorter than that of orlistat. Hence, shorter inhibitor residence times are known to translate into a faster dissociation of enzyme-inhibitor complexes in vivo [27, 28] and, in this case, delay but not completely inhibit the digestion of fats. In this respect, studies have reported that the presence of fatty acids and monoacylglycerol in the duodenal section of the intestine has more satiating effects than unhydrolyzed fats. Thus, compounds that reduce dietary fat absorption rates are valuable strategies for inducing satiety [29, 30]. For instance, dimethyl aminoethyl dodecyl ether, a non-hydrolysable lipid compound that delays, but does not inhibit, fat digestion by binding to pancreatic lipase, had a more satiating effect than compounds yielding unhydrolyzable fat in the intestine such as orlistat [31]. Similarly, researchers found that dietary supplementation of cell membranes from plants, animals, and bacteria decreased the rate of lipase/colipase catalyzed hydrolysis of TG, and when supplemented in combination with refined foods, the compounds suppressed food intake in rats by retardation of fat digestion [32]. Consistent with the results of this study, Ojeda et al. [33] evaluated the metabolic effect of a 25% canary seed infusion on rats under a hypercaloric diet for 45 days, where rats that received a whole canary seed infusion combined with a dietary change showed a reduction of 30% in TG values. Similarly, Almaraz et al. [34] found that both aqueous and hexane canary seed extracts successfully prevented metabolic syndrome indicators like hypertriglyceridemia. In agreement with this, we previously reported that canary seeds are predominantly rich in hydrophobic protein fractions called prolamins [15]. Subsequently, we fractionated the canary seed proteins and identified four peptides (e.g., VPPR, LADR, LSPR, and TVGPR) with high inhibitory activity towards pancreatic lipase. Taken together, it is possible that the dissociation of CSP-pancreatic lipase complexes played a critical role in reducing serum TG, adipose tissue deposition, and in the case of CSP-250, preventing weight gain by inducing satiety. While the health benefits of canary seeds have been previously reported, this is the first time that a proposed mechanism has been established using isolated canary seed peptides as bioactive compounds.

Obesity is also associated with an increased risk of non-alcoholic fatty liver disease (NAFLD), characterized by hepatic steatosis (intrahepatic fat of at least 5% of liver weight). Hepatic steatosis occurs when the uptake of fatty acid from plasma and the de novo fatty acid synthesis exceed the rate of fatty acid oxidation and metabolism, and its progression can lead to chronic liver disease, Type-2 diabetes, and cardiovascular disease [35,36]. Traditionally, treatments of NAFLD included pharmacological interventions (e.g., metformin, incretins, thiazolidinediones, anti-obesity drugs) and lifestyle modification (e.g., weight loss programs, change in diet, exercise) [37]. In addition, studies have also validated the efficacy of orlistat administration in treating NAFLD. For instance, a 6-month clinical trial involving 191 patients showed that 53% of the patients treated with orlistat improved their liver steatosis condition compared to routine treatments involving dietary and physical activity interventions [20]. In agreement with this, orlistat as well as CSP treatments were successful in the reduction of hepatic steatosis as well as reducing LDL/VLDL levels. Thus, the CSP-mediated effect on lipid synthesis, storage, and metabolism genes was studied. It was found that CSP suppressed the gene expression of FAS and ACC, transcription factors important in lipogenesis, as well as the LPL encoding gene, a transcription factor involved in lipid metabolism [36]. The role of FAS has been established as a critical multifunctional enzyme responsible for catalyzing the palmitate synthesis pathway and increasing de novo lipogenesis in the body, liver, and adipose tissue [38]. Likewise, ACC also promotes lipogenesis by mediating the catalysis of acetyl-CoA into malonyl-CoA, a two-carbon donor unit essential for the synthesis of palmitate fatty acids [39]. Recent studies have also established that increased upregulation of hepatic LPL is strongly associated with NAFLD due to its rate-controlling function in plasma triglyceride hydrolysis and incorporation into tissues [40,41]. In this study, CSP had no modulatory activity over transcription factors PPAR-α and ACO, genes involved in the metabolism and oxidation of fatty acids [42]. It is known that PPAR-α act as one of the master regulators responsible for translating nutritional and metabolic stimuli for fat oxidation genes such as Pdk4, Fgf21, ACO, and Cpt1α [38, 43]. At the same time, ACO functions as a rate-limiting enzyme involved in the first step of fatty acid peroxisomal 3-oxidation [42]. Overall, this study suggests that the anti-hepatic steatosis effect was likely attributed to a reduced systemic lipid uptake and deposition rather than enhanced lipid oxidation. Additionally, it was established that CSP has exceptional anti-hyperlipidemic properties and that at moderate concentration, it might prevent weight gain via retardation of lipid metabolism and hunger suppression. Indeed, hexane extracts from canary seeds decreased weight gain in the liver and reduced triglyceride deposition in hepatic tissue [44]. Furthermore, the extracts effectively decreased transcriptional factors associated with oxidative stress like SOD, CAT, and CSH.

In conclusion, administration of canary seed peptides (CSP) generated by commercial proteolysis suppressed metabolic consequences derived from obesity such as weight gain, hyperlipidemia, glucose tolerance, and hepatic steatosis of C57BL/6J mice fed a Western diet. Moderate supplementation of CSP (i.e., 250 mg/kg of body weight (for mice)) was more effective in preventing weight gain than the high CSP dose (i.e., 500 mg/kg of body weight (for mice)). Moreover, CSP supplementation reduced steatorrhea compared to the commercial drug orlistat. Likewise, CSP promoted a drop in fatty acid uptake gene, LPL, and fatty acid biosynthesis genes FAS and ACC, while unaffecting lipid oxidation genes PPAR-α and ACO. The regulation of these genes is related to a lower fat-derived fuel utilization and decreased bloodstream lipid uptake. Moreover, the results suggest that CSP's weight gain inhibition depends on a dual mechanism involving a delay in lipid metabolism and satiety to modulation. Supporting this theory, in vitro enzymatic analysis showed the rapid dissociation capacity of CSP-pancreatic lipase complexes and the critical role of its binding reversibility in reducing serum TG absorption rates and inducing satiety. Lastly, these results support the potential use of CSP as an effective nutraceutical for functional foods and as an anti-obesity treatment.

### REFERENCES

1. Haththotuwa, R. N.; Wijeyaratne, C. N.; Senarath, U. Worldwide epidemic of obesity. In Obesity and Obstetrics; Elsevier: Amsterdam, The Netherlands, 2020; pp. 3-8.
2. Jung, U. J.; Choi, M.-S. Obesity and its metabolic complications: The role of adipokines and the relationship between obesity, inflammation, insulin resistance, dyslipidemia and nonalcoholic fatty liver disease. Int. J. Mol. Sci. 2014, 15, 6184-6223.
3. Karczewski, J.; Sledzinska, E.; Baturo, A.; Jonczyk, I.; Maleszko, A.; Samborski, P.; Begier-Krasinska, B.; Dobrowolska, A. Obesity and inflammation. Eur. Cytokine Netw. 2018, 29, 83-94.
4. Seca, A. M.; Pinto, D. C. Overview on the antihypertensive and anti-obesity effects of secondary metabolites from seaweeds. Mar. Drugs 2018, 16, 237.
5. Kato, E. Bioactive compounds in plant materials for the prevention of diabetes and obesity. Biosci. Biotechnol. Biochem. 2019, 83, 975-985.
6. Chelliah, R.; Wei, S.; Daliri, E. B.-M.; Elahi, F.; Yeon, S.-J.; Tyagi, A.; Liu, S.; Madar, I. H.; Sultan, G.; Oh, D.-H. The role of bioactive peptides in diabetes and obesity. Foods 2021, 10, 2220.
7. Jang, E.; Moon, J.; Ko, J.; Ahn, C.; Lee, H.; Shin, J.; Park, C.; Kang, J. Novel black soy peptides with antiobesity effects: Activation of leptin-like signaling and AMP-activated protein kinase. Int. J. Obes. 2008, 32, 1161-1170.
8. Coronado-Caceres, L. J.; Rabadán-Chávez, G.; Mojica, L.; Hernandez-Ledesma, B.; Quevedo-Corona, L.; Lugo Cervantes, E. Cocoa Seed Proteins' (Theobroma cacao L.) Anti-Obesity Potential through Lipase Inhibition Using In Silico, In Vitro and In Vivo Models. Foods 2020, 9, 1359.
9. Mostafa, H.; Al-Ahbabi, N.; Adiamo, O. Q.; Mudgil, P.; Maqsood, S. Phoenix dactylifera L. seed protein hydrolysates as a potential source of peptides with antidiabetic and anti-hypercholesterolemic properties: An in vitro study. Food Biosci. 2022, 49, 101916.
10. Abdel-Aal, E.-S. M.; Hernandez, M.; Rabalski, I.; Hucl, P. Composition of hairless canary seed oil and starch-associated lipids and the relationship between starch pasting and thermal properties and its lipids. LWT 2020, 125, 109257.
11. Jurado Rodriguez, K. Y. Desarrollo de un producto alimenticio galletas nutritivas basadas en alpiste para personas diabéticas, con el fin de optimizar su salud en la ciudad de Guayaquil. Universidad de Guayaquil. Bachelor's Thesis, Facultad de Ciencias Administrativas, Universidad de Guayaquil, Guayaquil, Ecuador, 2015.
12. Rodríguez, C. D.; Hernández, L. H.; Larios, R. M.; Ramos, C. C. Phalaris canariensis (alpiste) como alimento funcional. Una alternativa para el tratamiento de la hipertensión y reducción de indice de masa corporal en adultos mayores. Investig. Cientifica 2020, 14, 156-161.
13. Valverde, M. E.; Orona-Tamayo, D.; Nieto-Rendón, B.; Paredes-López, O. Antioxidant and antihypertensive potential of protein fractions from flour and milk substitutes from canary seeds (Phalaris canariensis L.). Plant Foods Hum. Nutr. 2017, 72, 20-25.
14. Estrada-Salas, P. A.; Montero-Morin, G. M.; Martinez-Cuevas, P. P.; Gonzalez, C.; Barba de la Rosa, A. P. Characterization of antidiabetic and antihypertensive properties of canary seed (Phalaris canariensis L.) peptides. J. Agric. Food Chem. 2014, 62, 427-433.
15. Urbizo-Reyes, U.; Aguilar-Toali, J.; Liceaga, A. Hairless canary seeds (Phalaris canariensis L.) as a potential source of antioxidant, antihypertensive, antidiabetic, and antiobesity biopeptides. Food Prod. Processing Nutr. 2021, 3, 1-12.
16. Urbizo-Reyes, U.; Liceaga, A. M.; Reddivari, L.; Kim, K.-H.; Anderson, J. M. Enzyme kinetics, molecular docking, and in silico characterization of canary seed (Phalaris canariensis L.) peptides with ACE and pancreatic lipase inhibitory activity. J. Funct. Foods 2022, 88, 104892.
17. Passos, C. S.; Carvalho, L. N.; Pontes Jr, R. B.; Campos, R. R.; Ikuta, O.; Boim, M. A. Blood pressure reducing effects of Phalaris canariensis in normotensive and spontaneously hypertensive rats. Can. J. Physiol. Pharmacol. 2012 90, 201-208.
18. Bansal, A. B.; Al Khalili, Y. Orlistat. In StatPearls [Internet]; StatPearls Publishing: Treasure Island, FL, USA, 2020.
19. Cavaliere, H.; Floriano, I.; Medeiros-Neto, G. Gastrointestinal side effects of orlistat may be prevented by concomitant prescription of natural fibers (Psyllium mucilloid). Int. J. Obes. 2001, 25, 1095-1099.
20. Ye, J.; Wu, Y.; Li, F.; Wu, T.; Shao, C.; Lin, Y.; Wang, W.; Feng, S.; Zhong, B. Effect of orlistat on liver fat content in patients with nonalcoholic fatty liver disease with obesity: Assessment using magnetic resonance imaging-derived proton density fat fraction. Ther. Adv. Gastroenterol. 2019, 12, 1756284819879047.
21. Lunagariya, N. A.; Patel, N. K.; Jagtap, S. C.; Bhutani, K. K. Inhibitors of pancreatic lipase: State of the art and clinical perspectives. EXCLI J. 2014, 13, 897.
22. Liu, T.-T.; Liu, X.-T.; Chen, Q.-X.; Shi, Y. Lipase Inhibitors for Obesity: A Review. Biomed. Pharmacother. 2020, 128, 110314.
23. Tinline-Goodfellow, C. T.; West, D. W.; Malowany, J. M.; Gillen, J. B.; Moore, D. R. An acute reduction in habitual protein intake attenuates post exercise anabolism and may bias oxidation-derived protein requirements in resistance trained men. Front. Nutr. 2020, 7, 55.
24. Wu, G. Dietary protein intake and human health. Food Funct. 2016, 7, 1251-1265.
25. Schoenfeld, B. J.; Aragon, A. A. How much protein can the body use in a single meal for muscle-building? Implications for daily protein distribution. J. Int. Soc. Sports Nutr. 2018, 15, 10.
26. Yang, Z.; He, T.; Bumbie, G. Z.; Hu, H.; Chen, Q.; Lu, C.; Tang, Z. Effects of dietary crude protein levels on fecal crude protein, amino acids flow amount, fecal and ileal microbial amino acids composition and amino acid digestibility in growing pigs. Animals 2020, 10, 2092.
27. Oliveira, R. F.; Goncalves, G. A.; Inicio, F. D.; Koehnlein, E. A.; De Souza, C. G. M.; Bracht, A.; Peralta, R. M. Inhibition of pancreatic lipase and triacylglycerol intestinal absorption by a pinhao coat (Arancaria angustifolia) extract rich in condensed tannin. Nutrients 2015, 7, 5601-5614.
28. Lu, H.; Tonge, P. J. Drug-target residence time: Critical information for lead optimization. Curr. Opin. Chem. Biol. 2010, 14, 467-474.
29. Feltrin, K. L. The Role of the Free Fatty Acid, Lauric Acid, in Appetite Regulation and Its Potential as an Appetite-Suppressant. Ph.D. Thesis, University of Adelaide, Adelaide, Australia, 2008.
30. Feltrin, K. L.; Patterson, M.; Ghatei, M. A.; Bloom, S. R.; Meyer, J. H.; Horowitz, M.; Feinle-Bisset, C. Effect of fatty acid chain length on suppression of ghrelin and stimulation of PYY, GLP-2 and PP secretion in healthy men. Peptides 2006, 27, 1638-1643.
31. Mei, I.; Lindqvist, A.; Krabisch, L.; Rehfeld, J. F.; Erlanson-Albertsson, C. Appetite suppression through delayed fat digestion. Physiol. Behav. 2006, 89, 563-568.
32. Albertsson, P.-A.; Kbhnke, R.; Emek, S. C.; Mei, J.; Rehfeld, J. F.; Akerlund, H.-E.; Erlanson-Albertsson, C. Chloroplast membranes retard fat digestion and induce satiety: Effect of biological membranes on pancreatic lipase/co-lipase. Biochem. J. 2007, 401, 727-733.
33. Ojeda, L.; Da Ruiz, Y.; Martinez, F.; Odreman, R.; Torri, J.; Villegas, R.; Ybarra, L. P.; Machado, N. N. Effects of the seed of Phalaris canariensis and the change of diet on serum lipids in rats. Emir. J. Food Agric. 2021, 33, 287-292.
34. Almaraz, L. M.; Ramirez, J. L. E.; Santiago, J. Effects profiles of complete aqueous extract and hexane and aqueous fractions of Phalaris canariensis L. seeds on fructose-induced metabolic syndrome in rats. Int. J. Herb. Med. 2017, 5, 39-46.
35. Fabbrini, E.; Sullivan, S.; Klein, S. Obesity and nonalcoholic fatty liver disease: Biochemical, metabolic, and clinical implications. Hepatology 2010, 51, 679-689.
36. Nassir, F.; Rector, R. S.; Hammoud, G. M.; Ibdah, J. A. Pathogenesis and prevention of hepatic steatosis. Gastroenterol. Hepatol. 2015, 11, 167.
37. Richard, J.; Lingvay, I. Hepatic steatosis and Type 2 diabetes: Current and future treatment considerations. Expert Rev. Cardiovasc. Ther. 2011, 9, 321-328.
38. Nguyen, P.; Leray, V.; Diez, M.; Serisier, S.; Bloc'h, J. L.; Siliart, B.; Dumon, H. Liver lipid metabolism. J. Anim. Physiol. Anim. Nutr. 2008, 92, 272-283.
39. Strable, M. S.; Ntambi, J. M. Genetic control of de novo lipogenesis: Role in diet-induced obesity. Crit. Rev. Biochem. Mol. Biol. 2010, 45, 199-214.
40. Teratani, T.; Tomita, K.; Furuhashi, H.; Sugihara, N.; Higashlyama, M.; Nishikawa, M.; Irie, R.; Takajo, T.; Wada, A.; Horiuchi, K. Lipoprotein lipase up-regulation in hepatic stellate cells exacerbates liver fibrosis in nonalcoholic steatohepatitis in mice. Hepatol. Commun. 2019, 3, 1098-1112.
41. Birkenfeld, A. L.; Shulman, G. I. Nonalcoholic fatty liver disease, hepatic insulin resistance, and type 2 diabetes. Hepatology 2014, 59, 713-723.
42. Pérez-Echarri, N.; Pérez-Matute, P.; Marcos-Gómez, B.; Marti, A.; Martinez, J. A.; Moreno-Aliaga, M. J. Downregulation in muscle and liver lipogenic genes: EPA ethyl ester treatment in lean and overweight (high-fat-fed) rats. J. Nutr. Biochem. 2009, 20, 705-714.
43. Chow, J. D.; Lawrence, R. T.; Healy, M. E.; Dominy, J. E.; Liao, J. A.; Breen, D. S.; Byrne, F. L.; Kenwood, B. M.; Lackner, C.; Okutsu, S. Genetic inhibition of hepatic acetyl-CoA carboxylase activity increases liver fat and alters global protein acetylation. Mol. Metab. 2014, 3, 419-431.
44. Perez Gutierrez, R. M.; Madrigales Ahuatzi, D.; Horcacitas, M.d.C.; Garcia Baez, E.; Cruz Victoria, T.; Mota-Flores, J. M. Amelio-rative effect of hexane extract of Phalaris canariensis on high fat diet-induced obese and streptozotocin-induced diabetic mice. Evid.-Based Complementary Altern. Med. 2014, 2014, 145901.
45. Achouri, A.; L'Hocine, L.; Martineau-Côté, D.; Sirois, S.; Pitre, M.; Mason, E.; Abdel-Aal, E. M.; Hucl, P. Scale up fractionation of components from novel glabrous brown and yellow canary seeds (Phalaris canariensis L.) and techno-functional properties of the resulting protein isolates. Food Res. Int. 2020, 137, 109751.
46. Urbizo-Reyes, U.; Kim, K.-H.; Reddivari, L.; Anderson, J. M.; Liceaga, A. M. Oxidative Stress Protection by Canary Seed (Phalaris canariensis L.) Peptides in Caco-2 Cells and Caenorhabditis elegans. Nutrients 2022, 14, 2415.
47. Small, L.; Ehrlich, A.; Iversen, I.; Ashcroft, S. P.; Trost, K.; Moritz, T.; Hartmann, B.; Holst, J. J.; Treebak, J. T.; Zierath, J. R. Comparative analysis of oral and intraperitoneal glucose tolerance tests in mice. Mol. Metab. 2022, 57, 101440.
48. Kumar, M.; Lowery, R. G. A high-throughput method for measuring drug residence time using the transcreener ADP assay. SLAS DISCOVERY: Adv. Life Sci. RD 2017, 22, 915-922.
49. Adhyatmika, A.; Beljaars, L.; Putri, K. S.; Habibie, H.; Boorsma, C. E.; Reker-Smit, C.; Luangmonkong, T.; Guney, B.; Haak, A.; Mangnus, K. A. Osteoprotegerin is more than a possible serum marker in liver fibrosis: A study into its function in human and murine liver. Pharmaceutics 2020, 12, 471.
50. Smith, A.; Yu, X.; Yin, L. Diazinon exposure activated transcriptional factors CCAAT-enhancer-binding proteins α (C/EBPα) and peroxisome proliferator-activated receptor γ (PPARγ) and induced adipogenesis in 3T3-L1 preadipocytes. Pestic. Biochem. Physiol. 2018, 150, 48-58.
51. Siersbawk, M.; Varticovski, L.; Yang, S.; Baek, S.; Nielsen, R.; Mandrup, S.; Hager, G. L.; Chung, J. H.; Grentved, L. High fat diet-induced changes of mouse hepatic transcription and enhancer activity can be reversed by subsequent weight loss. Sci. Rep. 2017, 7, 40220.
52. Pfohl, M.; DaSilva, N. A.; Marques, E.; Agudelo, J.; Liu, C.; Goedken, M.; Slitt, A. L.; Seeram, N. P.; Ma, H. Hepatoprotective and anti-inflammatory effects of a standardized pomegranate (Punica granatum) fruit extract in high fat diet-induced obese C57BL/6 mice. hit. J. Food Sci. Nutr. 2021, 72, 499-510.
53. Kleiner, D. E.; Brunt E. M.; Van Natta, M.; Behling, C.; Contos, M. J.; Cummings, O. W.; Ferrell, L. D.; Liu, Y. C.; Torbenson, M. S.; Unalp-Arida, A.; et al. Design and validation of a histological scoring system for nonalcoholic fatty liver disease. Hepatology 2005, 41, 1313-1321.
54. Urbizo-Reyes, U.; Aguilar-Toala, J.; Liceaga, A. Hairless canary seeds (Phalaris canariensis L.) as a potential source of antioxidant, antihypertensive, antidiabetic, and antiobesity biopeptides. Food Prod. Process. Nur. 2021, 3, 1-12.
55. You, L.; Zhao, M.; Regenstein, J. M.; Ren, J. Changes in the antioxidant activity of loach (Misgurnus anguillicaudatus) protein hydrolysates during a simulated gastrointestinal digestion. Food Chem. 2010, 120, 810-816.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “substantially” can allow for a degree of variability in a value or range, for example, within 90%, within 95%, or within 99% of a stated value or of a stated limit of a range.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

1. A method of suppressing hunger in a human subject, who is obese or at risk for obesity, which method comprises administering at least about 20 mg/kg body weight/day of bioactive peptides comprising VPPR, LADR, LSPR, TVGPR, or any combination of two or more of the foregoing as a protein hydrolysate mixture, whereupon hunger in the subject is suppressed.

2. The method of claim 1, which comprises administering at least about 20 mg/kg body weight/day to about 40 mg/kg body weight/day of bioactive peptides as a protein hydrolysate mixture.

3. The method of claim 1, wherein the bioactive peptides are present in a low molecular weight (<3 kDa) peptide fraction obtained from canary seed (Phalaris canariensis L.).

4. The method of claim 2, wherein the bioactive peptides are present in a low molecular weight (<3 kDa) peptide fraction obtained from canary seed (Phalaris canariensis L.).

5. The method of claim 3, wherein the low molecular weight (<3 kDa) peptide fraction is obtained from canary seed (Phalaris canariensis L.) by a method comprising:
(i) suspending defatted canary seed flour in distilled water at a pH of about 10 and stirring;
(ii) centrifuging to obtain a protein-rich supernatant;
(iii) adjusting the pH of the protein-rich supernatant to about 4;
(iv) centrifuging to precipitate protein;
(v) suspending the protein in distilled water at a pH of about 8;
(iv) subjecting the suspended protein to proteolysis with Alcalase® (protease of Bacillus lichenformis);
(vii) terminating proteolysis;
(viii) centrifuging to obtain a whole protein hydrolysate (WPH) as a supernatant; and
(ix) either (a) ultrafiltering the supernatant using a <3 kDa cutoff membrane to collect a peptide fraction comprising bioactive peptides, freezing, and freeze-drying or (b) subjecting the WPH to simulated gastrointestinal digestion (SGD), terminating SGD, centrifuging to obtain a supernatant, ultrafiltering the supernatant using a <3 kDa cutoff membrane to collect a peptide fraction comprising bioactive peptides, freezing, and freeze-drying.

6. The method of claim 4, wherein the low molecular weight (<3 kDa) peptide fraction is obtained from canary seed (Phalaris canariensis L.) by a method comprising:
(i) suspending defatted canary seed flour in distilled water at a pH of about 10 and stirring;
(ii) centrifuging to obtain a protein-rich supernatant;
(iii) adjusting the pH of the protein-rich supernatant to about 4;
(iv) centrifuging to precipitate protein;
(v) suspending the protein in distilled water at a pH of about 8;
(vi) subjecting the suspended protein to proteolysis with Alcalase® (protease of Bacillus lichenformis);
(vii) terminating proteolysis;
(viii) centrifuging to obtain a whole protein hydrolysate (WPH) as a supernatant; and
(ix) either (a) ultrafiltering the supernatant using a <3 kDa cutoff membrane to collect a peptide fraction comprising bioactive peptides, freezing, and freeze-drying or (b) subjecting the WPH to simulated gastrointestinal digestion (SGD), terminating SGD, centrifuging to obtain a supernatant, ultrafiltering the supernatant using a <3 kDa cutoff membrane to collect a peptide fraction comprising bioactive peptides, freezing, and freeze-drying.

7. The method of claim 1, wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

8. The method of claim 2 wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

9. The method of claim 3, wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

10. The method of claim 4, wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

11. The method of claim 5, wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

12. The method of claim 6, wherein the bioactive peptides are administered as a protein hydrolysate mixture before or with a meal comprising protein, wherein the combined amount of bioactive peptides (as a protein hydrolysate mixture) and protein is about 0.33 g/kg body weight/meal to about 0.40 g/kg body weight/meal.

13. The method of claim 1, wherein glucose tolerance in the subject increases.

14. The method of claim 1, wherein weight gain in the subject is inhibited.

15. The method of claim 14, wherein the subject loses weight.
